(19) United States
(12) Patent Application Publication
(10) Pub. No.: US 2015/0374855 A1
(43) Pub. Date: Dec. 31, 2015

(54) ORAL CONTRAST MEDIUM FOR THE DIAGNOSTIC INVESTIGATION OF THE GASTROINTESTINAL TRACT BY MEANS OF MPI

(71) Applicant: TOPASS GMBH, Berlin (DE)

(72) Inventors: Katharina ROEPKE, Berlin (DE); Dietrich THORE, Berlin (DE); Etsuko HASEGAWA, Berlin (DE); Ulrich PISON, Berlin (DE); Michael GIERSIG, Kleinmachnow (DE)

(21) Appl. No.: 14/767,919

(22) PCT Filed: Feb. 20, 2014
(86) PCT No.: PCT/DE2014/100064

§ 371 Date: Aug. 14, 2015

(30) Foreign Application Priority Data
Feb. 20, 2013 (DE) 10 2013 101 686.3

(51) Int. Cl. A61K 49/18

(52) U.S. Cl. CPC A61K 49/18

(57) ABSTRACT

The invention relates to a pharmaceutically acceptable colloidal suspension, which comprises a microcapsule, which is enclosed by a container (“double container”), wherein the microcapsules comprise magnetic particles in a colloidal solution. The invention further relates to the use of said pharmaceutically acceptable colloidal suspension as an oral contrast medium for the diagnostic investigation of the gastrointestinal tract and the use of the contrast medium to diagnose proliferative diseases, inflammatory and autoimmune diseases, infectious diseases and hormonal and hereditary diseases in the gastrointestinal tract.

The invention relates to a pharmaceutically acceptable colloidal suspension, which comprises a microcapsule, which is enclosed by a container (“double container”), wherein the microcapsules comprise magnetic particles in a colloidal solution. The invention further relates to the use of said pharmaceutically acceptable colloidal suspension as an oral contrast medium for the diagnostic investigation of the gastrointestinal tract and the use of the contrast medium to diagnose proliferative diseases, inflammatory and autoimmune diseases, infectious diseases and hormonal and hereditary diseases in the gastrointestinal tract.

In the prior art, MPI (magnetic particle imaging) is known as a new imaging method that allows diagnostic methods in medicine through the use of suitable magnetic particles. Suitable contrast media are necessary for this procedure. With the aid of iron oxide nanoparticles, such a contrast medium is available by which an in vivo quantitative imaging is possible.

After iron oxide nanoparticles have been introduced into the bloodstream, for example, an angiographic image with high contrast may be successfully obtained, as MPI does not detect anatomical background signals. The sensitivity of the method allows real-time 3D imaging with clinically approved doses of iron oxide nanoparticles in small animals. In vivo experiments in mice using RESOVIST (Bayer Healthcare Pharmaceuticals) thus demonstrate the possibility of visualizing the flow of a bolus of particles through a beating mouse heart (8). Due to these capabilities, MPI has the potential to be an important tool in the field of imaging of the coronary arteries and the quantitative determination of perfusion in the myocardium.

MPI also promises advantages in the field of intestinal imaging over the existing non-invasive modalities CT and MRI (1). By means of oral administration of iron oxide nanoparticles as part of an oral contrast medium suspension, intestinal contents can be tagged. Due to the lack of anatomical background signal, an optimal contrast with the intestinal wall can be achieved with complete lining of the intestine, and a 3D “print image” can be created in which, for example, polyps are represented as “indentations.” Galenics is therefore of particular importance in relation to MPI—on the one hand, the stool must (as in colonography) have a relatively uniform concentration of contrast medium, additionally must therefore be ensured that the intestine is well-lined. This method is similar to the approaches to stool staining by contrast media (“fecal tagging”), which are already used with success to increase the contrast in CT and MRI colonography (4, 6). Due to the high sensitivity of MPI, a high signal can be achieved even with a low particle concentration. MPI enables the imaging of the entire intestinal volume, wherein recording is realized by means of sequential real-time measurements of intestinal sections. A software algorithm similar to the software that is available for CT-based screening (CT colonography) may then be used to detect polyps as the completely curable early stages of colon cancer.

Compared to CT colonography, the MPI method has the advantage that no harmful radiation is used. Firstly, acceptance of colonography as a screening method may thus be increased. Secondly, measurements are possible over a period of a few minutes with MPI, which would allow the representation of peristaltic movements. This dynamic additional information facilitates the localization of air bubbles and the identification of temporarily collapsed intestinal areas. In CT colonography, one attempts to obtain this information from a comparison of two images of the patient, one in the supine position and one in the prone position. With the aid of the additionally achieved dynamic information, disadvantages due to lack of intestinal cleansing can be compensated in the planned MPI process. The expectation also exists that, through the combination of high contrast and dynamic information in the MPI method, one can dispense with both the intestinal cleansing as well as the widening of the colon with air or CO2.

The primary advantage of the MPI method with respect to MRI colonography is the possibility of faster imaging, which leads to cost savings by means of short examination times. In the case of MRT, peristalsis must also be slowed down by the administration of Buscopan to avoid motion artifacts in imaging. In contrast, the high time resolution of the MPI method would allow real-time 3D imaging and thus, as already described in the previous paragraph, would dynamically deliver additional information with a simultaneously reduced overall scan time. The widening of the colon with gas or liquid required by MRT colonography could also be eliminated.

Due to the described benefits, it is expected that an MPI-based colorectal cancer screening will lead to improved compliance. Through this screening, colorectal cancer will be found at a stage where it is still completely curable. In later stages, when symptoms lead the patient to the doctor, the chance of survival is much lower and large costs for health systems are caused by low compliance.

In the prior art, patent applications have been filed for various methods of MPI; such as DE 10151778A1, “Method for determining the spatial distribution of magnetic particles.” However, in addition to the known device technology, a contrast medium is also an essential component of all diagnostic methods which seek to use MPI for imaging. Until now, the technological studies for the development of devices with MPI have been realized with Ferucarbotran (RESOVIST). This material was developed for magnetic resonance imaging in the 1990s (SCHERING). In WO 2007/000350, iron oxide nanoparticles are disclosed as a contrast medium for MPI which may be used in an injection into a blood vessel, preferably a vein. Intestinal screening tests utilizing MPI are further described in the prior art (2).

However, no suitable contrast media for oral administration have been described in the prior art which could be used for the diagnostic investigation of the gastrointestinal tract.

The object of the invention was therefore to provide a contrast medium for oral administration which may be used for diagnostic investigation of the gastrointestinal tract by means of MPI.

It was quite surprising that an orally ingestible contrast medium can be provided for the diagnostic investigation of the gastrointestinal tract, wherein said contrast medium comprises a pharmaceutically acceptable colloidal suspension comprising a pharmaceutically acceptable coated container which is designed as a “double container” having a diameter of 0.6-300 μm and of which the inner container is designed as a microcapsule having a colloidal solution or suspension in which the magnetic particles are contained. The double container preferably has a diameter of 5-100 μm. The magnetic particles are thereby incorporated in a biodegradable matrix, the nature of which permits Brownian motion. Said matrix is therefore characterized by a suitable viscosity. The viscosities measured in millipascal seconds may be between 1 and 2000. The viscosity can be adjusted, for example, by a glycerol-water solution, sugar water solution, oils or polymer blends. A person of ordinary skill in the art can test in the form of preliminary trials which biodegradable materials permit the Brownian motion of the magnetic particles. One skilled in the art need not perform an inventive step in this regard, but rather can easily test biodegradable materials known from the prior art by means of routine experimentation. The outer and inner containers are filled with a biodegradable matrix. If the matrix has a viscosity in the range of 0.5-100 mPa*s (millipascal seconds), the Brownian motion of the magnetic nanoparticles is retained. As a new imaging method, MPI requires a contrast medium for imaging. For known intravenous administration, various contrast media are available in the form of iron oxide nanoparticle preparations. Until the occurrence of this invention, there has been no suitable contrast medium for dosage in oral form, as the composition and preparation of magnetic nanoparticles had not yet been designed under the special requirements of the anatomical and physiological conditions of the gastrointestinal tract in light of MPI. Accordingly must be considered in the oral application of magnetic particles for the diagnosis of pathological changes in the gastrointestinal tract (i) anatomical and (ii) physiological conditions, in order to ensure informative imaging of the anatomical structures or physiological relationships in the respective sections of the gastrointestinal tract. It is to the credit of the inventors that a technical teaching is provided in which the orally administered contrast medium for the diagnostic investigation of the gastrointestinal tract can be designed such that the concentration of the magnetic particles can be adjusted in the various sections of the gastrointestinal tract to the specific magnetic susceptibility of the given particle.

It is furthermore surprising that the medium according to the invention is toxicologically harmless, as the lowest possible systemic absorption takes place through the mucosa of the intestinal tract through the choice of a suitable diameter for the oral contact medium. It was quite surprising that the stated requirements can be fulfilled through a coated container, which is designed as a “double container.” During the passage through the gastrointestinal tract, this double container ensures firstly that the magnetic particles are present in a biodegradable matrix in the inner container, wherein the biodegradable matrix allows Brownian motion, wherein the outer container allows the passage of these particles through the stomach without damage. The inner container is a microcapsule, i.e. a hollow body, which can accommodate the magnetic particles in a colloidal solution. The preparation of such microparticles is generally well-known and can be undertaken in a variety of ways, so that one of ordinary skill in the art can prepare them at any time. Known methods for the production of suitable microparticles may be interfacial polymerization, phase separation and coacervation, solvent evaporation techniques, spray methods, drying methods and centrifugation methods.

In the inner container, the magnetic particles are integrated in a biodegradable matrix such that the Brownian motion of these particles is possible. This has the advantage that by using appropriate sequences, a higher signal yield during detection of MPI signals is possible. As one skilled in the art can easily find such biodegradable materials, the technical teaching of the invention is sufficiently disclosed. Particularly good signal yields are achieved if the particles in the inner container can be freely rotated and displaced such that they can align their magnetic preferential directions corresponding to a preferred direction given by the sequence. The particles, in a container which permits Brownian motion, thus have a uniform magnetization behavior and therefore better MPI signal behavior under an appropriate sequence than such particles as are placed in a container which prevents Brownian motion. Crucial here is that the particles in the inner container can be freely rotated and displaced such that they can align their magnetic preferential directions corresponding to a preferred direction given by the sequence. Particles in a container which hinders Brownian motion cannot complete an alignment through rotation and displacement, for which reason the magnetic preferred directions are randomly distributed. The particles, in a container which permits Brownian motion, thus have a uniform magnetization behavior and therefore better MPI signal behavior under an appropriate sequence than such particles as are placed in a container which prevents Brownian motion.

Unger first proposed in 1991 (see WO 1 992/01 751 4 A1) the use of homogeneous aqueous suspensions of microcapsules as oral contrast media for CT, wherein the microcapsules have a low density (7). The microcapsules preferably comprise a biocompatible and toxicologically harmless synthetic polymer or copolymer. With regard to the materials of the microcapsules (biocompatible, metabolically stable and slowly biodegradable natural and synthetic polymers or copolymers, for instance cyanoacrylates, polylactides) and their preparation, reference is made inter alia to this document. The patent of Pison et al., WO 2007093451 A2, discloses preferred materials for the microcapsules (5).

The outer diameter of the inner container, i.e. the size of the microcapsules, is preferably from 0.5 to 50 μm, preferably between 0.3 pm and 10 μm. The microcapsules preferably do not have a uniform outer diameter, but rather a size distribution in terms of a normal distribution between 0.5 and 50 μm, preferably between 3 to 5 μm, especially preferably between 0.2 and 5 μm is present. A non-uniform size distribution allows for better lining and therefore visualization of the intestinal tube. Optionally, a greater variance of the size distribution may be desirable.

The inner container is surrounded by an outer container. The outer container protects the inner container and its contents. During the gastrointestinal passage, a release of the magnetic nanoparticles located in the container in a biodegradable matrix takes place after passage through the stomach, preferably in the ileum (although not necessarily there, as a release in the duodenum may also take place through surface structures and material selection), whereby the container is protected from the acid of the stomach. The pharmaceutical preparations according to the invention which may play a role here can be manufactured according to conventional methods such as compression, immersion or fluidized bed processes or pan coating, and may contain excipients and other customary auxiliaries, such as starch, for instance potato, corn or wheat starch, cellulose or derivatives thereof, in particular microcrystalline cellulose, silicon dioxide, various sugars such as lactose, magnesium carbonate and/or calcium phosphates. The coating solution usually consists of sugar and/or starch syrup, and contains gelatin, gum arabic, polyvinylpyrrolidone, synthetic cellulose esters, surfactants, plasticizers, and/or pigments, and similar additives according to the prior art. For preparation of the dosage forms may be used any of the conventional flow regulators, lubricants or glidants such as magnesium stearate and release agents.

Surprisingly, the design of the oral contrast medium as a double container, wherein an inner container or microcapsule is enclosed by a further container, wherein the outer container has a diameter from 6-300 μm, preferably between 5-100 μm, leads to a particularly good MPI signal behavior. Particularly good signals are obtained especially at a size of the inner container between 3-10 μm.

It is especially advantageous if the outer shell of the double container is enteric. It is hereby achieved that the colloidal solution is stabilized, whereby the inner container is released only after passage through the upper gastrointestinal tract.

In a further preferred embodiment of the invention, it is provided that the inner container or microcapsule comprises at least three magnetic single domain particles of a size between 3 nm-20 nm, which are present in a biodegradable matrix, or else at least two polycrystalline magnetic iron oxide cores with a diameter of 10-300 nm, likewise in a biodegradable matrix, are present. A person of ordinary skill in the art will here select the biodegradable matrix such that it allows Brownian motion. The feature of a biodegradable matrix which allows Brownian motion does not relate in this regard to an object set out before the person skilled in the art which can only be achieved through an inventive step; rather, this relates here to a functional feature of the invention which the person skilled in the art can implement without an inventive step. Appropriate biodegradable matrices are known to a person skilled in the art, or can be identified through routine investigation. It was surprising that the oral contrast medium achieves the object according to the invention particularly well when it has at least three magnetic single domain particles of a size between 3-20 nm, or else at least two polycrystalline magnetic iron oxide cores with a diameter of 10 nm to 300 nm. The parameter ranges mentioned do not constitute an arbitrary range, but rather reflect the diameter size which allows a surprisingly good implementation of the object according to the invention. It was surprising that the described sizes led to especially positive results in the case of oral contrast media for the diagnostic investigation of the gastrointestinal tract by means of MPI. This means that diseases of the gastrointestinal tract can be detected particularly well. This relates in particular to proliferative diseases, inflammatory and autoimmune diseases, infectious diseases, hormonal disorders and hereditary diseases. Particularly preferred are colorectal cancer screening tests.

In a preferred embodiment of the invention, the biodegradable matrix within the inner container, i.e. the microcapsule, is a synthetic polymer or copolymer, a starch or a derivative thereof, a dextran or a derivative thereof, a cyclodextran or a derivative thereof, a fatty acid, a polysaccharide, a lecithin or a mono-, di- or triglyceride or a derivative or a mixture thereof. Even if the mentioned substances refer to those which are already known to a person skilled in the art, it was surprising that these substances led to especially good results, particularly in view of orally applied contrast media for the diagnostic investigation of the gastrointestinal tract by means of MPI. It was surprising that, from the very large number of possible compounds, those named led in particular to especially good results. It was not obvious that these compounds known to a person skilled in the art would enable especially good early diagnosis in particular of colorectal cancer in the case of a contrast medium which is taken orally for MPI in connection with the diagnostic investigation of the gastrointestinal tract. The combination of the compounds mentioned and the preferred size of the magnetic particles results in a surprisingly good contrast, which makes it possible to diagnose even the smallest changes in the gastrointestinal tract.

In a further preferred embodiment of the invention, the magnetic particles comprise magnetite or maghemite, or mixtures thereof, or rare earth metal ions, transition metal ions and/or alloys thereof. It was most especially surprising that the combination of said magnetic particles in connection with the claimed size of the magnetic single domain particles or the polycrystalline, magnetic iron oxide cores in connection with the preferred biodegradable matrices led to surprisingly good results in the diagnosis of the gastrointestinal tract, in particular in the case of colorectal cancer. Particularly in the early diagnosis of diseases of the gastrointestinal tract, surprisingly good results can be achieved by means of MPI in connection with the orally administered contrast medium according to the invention. This was surprising, because many individual components of the teaching according to the invention were known. It was, however, not known that the combination of the known media and parameters leads to surprisingly good results in the diagnosis of the gastrointestinal tract. It was surprising that especially proliferative diseases, inflammatory and autoimmune diseases, infectious diseases, hormonal disorders and hereditary diseases can be diagnosed in a surprisingly early stage.

The mentioned surprising results can be again improved if the iron concentration of the magnetic particles in the inner container is 0.01 mmol/L to 0.1 mol/L, preferably 1 mmol/L to 100 mmol/L. In particular, the concentration of 1 mmol/L to 100 mmol/L results in that the object according to the invention is particularly well achieved. Particularly the combination of the features of the size of the double container between 5 μm and 100 μm, the advantageous size of the magnetic particles (3 nm-20 nm for single magnetic particles or 10 nm to 300 nm for polycrystalline, magnetic iron oxide cores) and the preferred iron oxide concentration of the magnetic particles (magnetite or maghemite) of 1 mmol/L to 100 mmol/L, wherein at least three magnetic single domain particles or at least two polycrystalline, magnetic iron oxide cores are incorporated in the biodegradable matrix (viscosity in the range of 0.5 to 100 mPa*s (millipascal seconds)), leads in combination with the preferred claimed biodegradable matrices to an especially good result in the diagnostic investigation of gastrointestinal diseases which are selected from the group comprising proliferative diseases, inflammatory and autoimmune diseases, infectious diseases, hormonal disorders and hereditary diseases, especially in colorectal cancer screening. Through the combination of the mentioned preferred technical features, a particularly good effect is achieved in the diagnosis of the gastrointestinal tract by means of MPI which is greater than a potential additive effect of the individual preferred technical features. This means that the combination of the preferred features leads to a particularly good result in early diagnosis, since the individual components for achieving the object according to the invention interact synergistically.

The teaching according to the invention therefore also relates to a method for the diagnostic investigation of the gastrointestinal tract by means of MPI, in which the oral contrast medium according to the invention is used. The invention further relates to a pharmaceutically acceptable colloidal suspension comprising a pharmaceutically acceptable coated container which is designed as a “double container” having a diameter of 0.6 μm-300 μm, preferably between 5 μm and 100 μm, the inner container of which contains magnetic particles incorporated in a biodegradable matrix, the nature of which permits Brownian motion.

The invention relates in a further aspect to the use of the mentioned pharmaceutically acceptable colloidal suspension or the oral contrast medium for the production of a diagnostic agent for use in imaging through detection of magnetic particles (MPI) for the diagnosis of a disease in the gastrointestinal tract which is selected from the group comprising proliferative diseases, inflammatory and autoimmune diseases, infectious diseases, hormonal disorders and/or hereditary diseases.

In a preferred embodiment, it is provided that the proliferative disease is selected from the group comprising: a tumor, a precancerous condition, a dysplasia, a neuroendocrine tumor, endometriosis and/or metaplasia.

In a further preferred embodiment of the invention, the inflammatory and autoimmune disease is selected from the group comprising inflammatory bowel disease, Crohn's disease and/or systemic lupus erythematous.

In a further preferred embodiment, the infectious disease is selected from the group comprising a parasitic disease, a bacterial disease and/or a viral disease.

In a further preferred embodiment, the hormonal disorder is selected from the group comprising a disorder of the glucose metabolism, lipid metabolism, protein metabolism, sexual development and reproduction, water-salt balance, growth and/or cell formation.

In a further preferred embodiment of the invention, the hereditary disease is selected from the group comprising an autosomal recessive, autosomal dominant, a gonosomal and/or mitochondrial and/or extra-chromosomal hereditary disease or a disease that can be traced back to a genetic predisposition.

With the inventive pharmaceutically acceptable colloidal suspension and the orally applied contrast medium is carried out, for example, various tests by means of MPI. The focus of these tests is a diagnostic investigation of the gastrointestinal tract. Proliferative diseases, inflammatory and autoimmune diseases, infectious diseases, hormonal disorders and/or hereditary diseases can hereby be detected with high certainty and at a very early stage.

Microcapsules having a diameter between 3-10 μm are loaded with magnetic particles such that they have at least three magnetic single domain particles of a size between 3-20 nm, or else at least two polycrystalline magnetic iron oxide cores with a diameter of 10 nm to 300 nm. These microcapsules are enclosed in a container such that the double container has a size of 5 μm to 100 μm. The magnetic particles (at least having magnetite or maghemite) have an iron concentration within the inner container (microcapsule) of 1 mmol/L to 100 mmol/L. The biodegradable matrix of the container comprises synthetic polymers or copolymers, a starch or a derivative thereof, a dextran or a derivative thereof, a cyclodextran or a derivative thereof, a fatty acid, a polysaccharide, a lecithin or a mono-, di- or triglyceride or a derivative thereof or mixture thereof. Using this oral contrast medium, proliferative diseases, inflammatory and autoimmune diseases, infectious diseases, hormonal disorders or hereditary diseases of the gastrointestinal tract can be diagnosed early and very confidently. Particularly good results are achieved in colorectal cancer screening.

### LITERATURE

1. Achiam M P, Løgager V B, Chabanova E, Eegholm B, Thomsen H S, and Rosenberg J. Diagnostic accuracy of MR colonography with fecal tagging. Abdom Imaging 34: 483-490, 2009.
2. BORGERT J, Gleich B, Kuhn M H, RAHMER J E, and Schmale I. Colon Screening by using magnetic particle imaging. Google Patents, 2012.
3. Briel A, Gleich B, Weizenecker J, Rohrer M, Weinmann H J, Pietsch H, Lawaczeck R, Rothe M, and Thomsen J. Compositions containing magnetic iron oxide particles, and use of said compositions in magnetic particle imaging. Google Patents, 2007.
4. Frentz S M, and Summers R M. Current Status of CT colonography. Academic Radiology 13: 1517-1531, 2006.
5. Pison U. Multimodal imaging using a three compartment polymer nanoparticle with cell specificity. Google Patents, 2007.
6. Thornton E, Morrin M M, and Yee J. Current Status of MR Colonography 1. Radiographics 30: 201 -218, 2010.
7. Unger E C. Low density microspheres and their use as contrast agents for computed tomography. Google Patents, 1992.
8. Weizenecker J, and al. e. Three-dimensional real-time in vivo magnetic particle imaging. Physics in Medicine and Biology 54: L1-L10, 2009.

1. An oral contrast medium for the diagnostic investigation of gastrointestinal tract via magnetic particle imaging (MPI),
comprising
a container having a diameter of 0.6-300 μm, and
a microcapsule enclosed by the container, wherein the microcapsule comprises magnetic particles in a colloidal solution.

2. The contrast medium according to claim 1,
wherein
the container has a diameter of 5-100 μm.

3. The contrast medium according to claim 1,
wherein
the microcapsule comprises:
at least three magnetic single domain particles of a size between 3 nm-20 nm, or at least three polycrystalline magnetic iron oxide cores with a diameter of 10 nm to 300 nm, and
a biodegradable matrix.

4. The medium according to claim 1,
wherein
the diameter of the microcapsules is 0.5-50 μm.

5. The medium according to claim 4,
wherein
the diameter of the microcapsules is between 3 μm-10 μm.

6. The medium according to claim 3,
wherein
the biodegradable matrix within the microcapsule comprises a synthetic polymer or co-polymer, a starch, a dextran, a cyclodextran, a fatty acid, a polysaccharide, a lecithin or a mono-, di- or triglyceride and/or derivatives or mixtures thereof.

7. The medium according to claim 1,
wherein the magnetic particles comprise
(i) magnetite, maghemite or mixtures thereof, the magnetic particles and
(ii) rare earth metal ions, transitional metal ions or alloys.

8. The medium according to claim 1,
wherein the magnetic particles have an iron concentration of 0.01 mmol/L to 1 mol/L.

9. Method for production of a diagnostic agent comprising the medium according to claim 1 for use in magnetic particles imaging (MPI) for diagnosing a disease in the gastrointestinal tract selected from the group consisting of proliferative diseases, inflammatory and autoimmune diseases, infectious diseases, hormonal disorders hereditary diseases and combinations thereof.

10. The method of claim 15, wherein the gastrointestinal tract of the patient is investigated for a proliferative disease comprising: a tumor, a precancerous condition, a dysplasia, a neuroendocrine tumor, endometriosis and/or metaplasia.

11. The method of claim 15, wherein the gastrointestinal tract of the patient is investigated for an inflammatory or autoimmune disease comprising inflammatory bowel disease, Crohn's disease and/or systemic lupus erythematosus.

12. The method of claim 15, wherein the gastrointestinal tract of the patient is investigated for an infectious disease comprising a parasitic disease, a bacterial disease and/or a viral disease.

13. The method of claim 15, wherein the gastrointestinal tract of the patient is investigated for a hormonal disorder comprising a disorder of the glucose metabolism, lipid metabolism, protein metabolism, sexual development and reproduction, water-salt balance, growth and/or cell formation.

14. The method of claim 15, wherein the gastrointestinal tract of the patient is investigated for a hereditary disease comprising an autosomal recessive, autosomal dominant, a gonosomal and/or mitochondrial and/or extra-chromosomal hereditary disease or a disease that can be traced back to a genetic predisposition.

15. A method for investigating a gastrointestinal tract of a patient comprising:
orally administering a contrast medium to the patient comprising
a container having a diameter of 0.6-300 μm, and
a microcapsule enclosed by the container, wherein the microcapsule comprises magnetic particles in a colloidal solution in a pharmaceutically acceptable colloidal suspension, and
subjecting the gastrointestinal tract of the patient to magnetic particle imaging (MPI).

16. The method of claim 15, wherein the gastrointestinal tract of the patient to MPI to detect a disease select from the group consisting of proliferative diseases, inflammatory and autoimmune diseases, infectious diseases, hormonal disorders and hereditary diseases.

17. The method of claim 15,
wherein
the container has a diameter of 5-100 μm.

18. The method of claim 15,
wherein the microcapsule comprises:
at least three magnetic single domain particles of a size between 3 nm-20 nm, or at least three polycrystalline magnetic iron oxide cores with a diameter of 10 nm to 300 nm, and
a biodegradable matrix.

19. The method of claim 15, wherein the diameter of the microcapsules is 0.5-50 μm.

20. The medium according to claim 8, wherein the magnetic particles have an iron concentration of from 1 mmol/L to 100 mmol/L.
